(19) United States
(12) Patent Application Publication
(10) Pub. No.: US 2025/0303135 A1
(43) Pub. Date: Oct. 2, 2025

(54) BI-DIRECTIONAL BLOOD PUMPS AND ONE-WAY FILTER TRAPS AND SYSTEMS INCLUDING THE SAME

(71) Applicants: Keshava Rajagopal, Berwyn, PA (US); THE TEXAS A&M UNIVERSITY SYSTEM, College Station, TX (US)

(72) Inventors: Keshava Rajagopal, Berwyn, PA (US); Kumbakonam Rajagopal, College Station, TX (US); Shivanand Pattanshetti, College Station, TX (US); Darbha Swaroop, College Station, TX (US)

(21) Appl. No.: 18/864,254

(22) PCT Filed: May 9, 2023
(86) PCT No.: PCT/IB2023/054809

§ 371 Date: Nov. 8, 2024

Related U.S. Application Data
(60) Provisional application No. 63/339,969, filed on May 9, 2022.

(51) Int. Cl. A61M 60/109; A61M 60/221; A61M 60/531

(52) U.S. Cl. CPC A61M 60/109; A61M 60/221; A61M 60/531; A61M 2205/3334; A61M 2205/3365

(57) ABSTRACT

A blood pump system switchable between a first flow direction and a second flow direction includes a filter trap and a blood pump. The blood pump has an impeller configured to rotate at a predetermined speed in a first rotary direction to generate fluid flow in the first flow direction at a first pressure and a first flowrate. The impeller is configured to rotate in a second rotary direction at the predetermined pump speed to generate fluid flow in the second flow direction at a second pressure and a second flowrate. The first flowrate and the first pressure are different from the second flowrate and the second pressure with the impeller operating at the predetermined pump speed. The filter trap includes a one-way valve configured to remain closed with fluid flow the first direction and to open with fluid flow in the second direction.

### TECHNICAL FIELD

The present disclosure relates generally to blood pumps, and more specifically, to bi-directional blood pumps and one-way filter traps.

### BACKGROUND

When treating a patient, it is sometimes necessary to generate blood flow using artificial pumps. In a perfusion arrangement, blood is pumped from one compartment of the circulation to another a blood pump. Typically, blood is pumped in an antegrade flow, meaning in the natural direction of blood flow. This may be in series or in parallel to native blood flow, depending upon the pump inflow and outflow connections. The blood may then be subjected to some kind of processing or treatment within an artificial circuit before being returned to circulate within the patient. In a retrograde flow, blood is pumped opposite to the natural direction of blood flow. Regardless of the direction of flow, it is necessary for the blood pump to approximately match the pressures of the vein and artery to which the blood pump is fluidly coupled. Failure to do so could result in damage to a blood vessel. Normal pressure within a systemic vein is typically in a range of 2-10 mmHg, and normal pressure within an artery is typically in a range of 100-140 mmHg, with mean pressures in a range of 65-95 mmHg.

Perfusion entails pumping fluid through the body with a physiologically normal flow to provide gas exchange and nutrient delivery to tissue and organs. Retroperfusion entails pumping fluid through the body with a physiologically abnormal flow. Prior methods for performing perfusion and retroperfusion are complicated and are accompanied by the risk of human error. For example, many blood pumps only operate in a single direction. In order to change the direction of flow (e.g., from antegrade flow to retrograde flow), leads to the pump must be disconnected and reconnected. Current continuous-flow pumps that are capable of operating bi-directionally require the pump speed to be changed between antegrade and retrograde flow to account for the normal range pressure differential between veins and arteries. An additional concern when switching from antegrade flow or perfusion to retrograde flow or retroperfusion is the possibility of manipulation-induced technical complications, most importantly bleeding and thrombus formation.

### SUMMARY OF THE INVENTION

This disclosure is directed to bi-directional blood pump systems for perfusion and retroperfusion. The bi-directional blood pump systems may operate at the same pump speeds for both perfusion and retroperfusion. The bi-directional blood pump systems may include a filter trap, an alarm, a gas exchanger, a vacuum, or a heat exchanger.

In an aspect of the present disclosure, a blood pump includes a pump housing and an impeller. The pump housing has a first connector configured as a fluid inlet into the pump housing in the first flow direction and as a fluid outlet from the pump housing in the second flow direction, and a second connector configured as a fluid inlet into the pump housing in the second flow direction and as a fluid outlet from the pump housing in the first flow direction. The impeller is disposed within the pump housing. The impeller is configured to rotate at a predetermined speed in a first rotary direction to generate fluid flow in the first flow direction at a first pressure and a first flowrate. The impeller is configured to rotate in a second rotary direction, opposite the first rotary direction, at the predetermined pump speed to generate fluid flow in the second flow direction at a second pressure and a second flowrate. The first flowrate is greater than the second flowrate and the first pressure is greater than the second pressure.

In aspects, the impeller includes a body and a vane. The body may have a first segment and a second segment. The vane may extend radially outward from the body and helically wrapped about the second segment of the body. The vane may have a rake such that radially outer parts of the vane are farther from the first segment than radially inner parts of the vane. The vane may helically wrap about the second segment of the body with a variable pitch such that parts of the vane closer to the first segment have a lesser pitch than parts of the vane farther from the first segment. The first segment may be substantially egg-shaped, and the second segment is frustoconically shaped. A product of the first flowrate and the first pressure may be different from a product of the second flowrate and the second pressure. The product of the first flowrate and the first pressure may be greater than the product of the second flowrate and the second pressure.

In particular aspects, the first pressure of the fluid flow generated by the blood pump is greater than the second pressure. The first flowrate of the fluid flow generated by the blood pump may be greater than the second flowrate. The second flowrate may between 30% and 50% of the first flowrate. The first pressure may be in the range of 100 mmHg to 140 mmHg. The first flowrate may be in the range of 2.8 L/min. to 3.5 L/min. The second pressure may be in the range of 2 mmHg to 10 mmHg. The second flowrate may be 1.2 L/min. to 1.5 L/min.

In another aspect of the present disclosure, a filter trap includes a housing, a first trap connector, a second trap connector, a perforated wall, and a one-way valve. The perforated wall is disposed within the housing and defines an interior chamber and an exterior chamber within the housing. The one-way valve segregates the interior chamber into an inlet section and a trap house.

In certain aspects, the perforated wall defines a plurality of perforations. The perforations may have a diameter in the range of 50 μm to 100 μm. The one-way valve may be configured to remain closed when fluid flows through the pump housing in the first flow direction and to open when fluid flows through the pump housing in the second flow direction. The one-way valve may have two flexible members attached to the perforated wall. The two flexible members may be configured to engage each other in a closed position and separate from each other in an open position. The two flexible members are self-biased towards the closed position. The perforated wall may include a first perforated portion and a second perforated portion. The first perforated portion may be separated from the second perforated portion by an unperforated portion. The two flexible members may each have a wall engagement portion configured to engage the unperforated portion when the two flexible members are in the open position. When each flexible member is in the open position and engaged with the unperforated portion of the perforated wall, each flexible member may define a trap room with perforated wall. The second perforated portion may be segregated from the trap house within a respective trap room by the flexible members. Fluid flow through the filter trap in the second flow direction may be impeded from flowing through the second perforated portions by the flexible members engaged with the unperforated portion. The perforated wall may include a third perforated portion configured to place the exterior chamber and the inlet section in direct fluid communication. The filter trap may include a third trap connector in direct fluid communication with the trap house. The third trap connector may be configured to fluidly couple the filter trap to a vacuum.

In another aspect of the present disclosure, a blood pump system including any blood pump detailed herein and any filter trap detailed herein. The blood pump system is switchable between a first flow direction and as second flow direction includes a filter trap and a blood pump. The blood pump system includes a pump housing. The pump housing has a first connector configured as a fluid inlet into the pump housing in the first flow direction and as a fluid outlet from the pump housing in the second flow direction, and a second connector in fluid communication with the filter trap configured as a fluid inlet into the pump housing in the second flow direction and as a fluid outlet from the pump housing in the first flow direction. The blood pump housing has an impeller disposed within the pump housing. The impeller is configured to rotate at a predetermined speed in a first rotary direction to generate fluid flow in the first flow direction at a first pressure and a first flowrate. The impeller is configured to rotate in a second rotary direction, opposite the first rotary direction, at the predetermined pump speed to generate fluid flow in the second flow direction at a second pressure and a second flowrate. A product of the first flowrate and the first pressure is different from a product of the second flowrate and the second pressure.

In certain aspects, the blood pump system includes a first tube fluidly coupled to the pump housing about the first connector. The first tube may be configured to fluidly couple to a first blood vessel of the human body. The blood pump system may include a second tube fluidly coupled to the pump housing about the second connector and configured to fluidly couple to a second blood vessel of the human body.

In aspects, the blood pump system includes a gas exchanger fluidly coupled with the blood pump. The blood pump system may include a heat exchanger fluidly coupled to the pump housing. The heat exchanger may be configured to maintain a desired temperature of a fluid. The blood pump system may include a vacuum. The vacuum may be fluidly coupled to the filter trap.

In another aspect of the present disclosure, a kit includes a blood pump system sealed in sterile packaging. The kit may include any of the blood pump systems described herein. Any of blood pumps, blood pump systems, and kits described herein may be operated in accordance with any method described herein.

In another aspect of the present disclosure, a method of pumping blood in a first direction and a second direction includes inserting a first tube into a first blood vessel of a patient, the first tube is fluidly coupled to a blood pump. Inserting a second tube into a second blood vessel of a second blood vessel of the patient, the second tube is fluidly coupled to the blood pump. Operating the blood pump at first operational pump speed such that the blood flows through the blood pump in a first flow direction into the first blood vessel of the patient at a first pressure and at a first flowrate. Operating the blood pump at the first operational pump speed such that blood flows through the pump in a second flow direction into the second blood vessel at a second pressure and a second flowrate. A product of the first pressure and the first flowrate is different from a product of the second pressure and the second flowrate.

Further, to the extent consistent, any of the embodiments or aspects described herein may be used in conjunction with any or all of the other embodiments or aspects described herein.

### BRIEF DESCRIPTION OF THE DRAWINGS

Various aspects of the present disclosure are described hereinbelow with reference to the drawings, which are not necessarily drawn to scale, which are incorporated in and constitute a part of this specification, wherein:

FIG. 1 is an isometric view of a bi-directional blood pump, according to aspects of the disclosure;

FIG. 2 is sectioned side view of the bi-directional blood pump of FIG. 1, according to aspects of the disclosure;

FIGS. 3A-3E are views of an impeller, according to aspects of the disclosure;

FIG. 4 is an isometric view of a filter trap, according to aspects of the disclosure;

FIG. 5 is an exploded assembly of the bi-directional blood pump of FIG. 4, according to aspects of the disclosure; and

FIG. 6 is an isometric view of an interior of the filter trap of FIG. 4 with the one-way valve in a closed position;

FIG. 7 is an isometric view of an interior of the filter trap of FIG. 4 with the one-way valve in an open position;

FIG. 8 is a view of a blood pump system in accordance with aspects of the present disclosure;

FIG. 9 is a view of a blood pump kit in accordance with aspects of the present disclosure; and

FIG. 10 is a flowchart showing a method of pumping blood in accordance with aspects of the present disclosure.

### DETAILED DESCRIPTION

The present disclosure will now be described more fully hereinafter with reference to example embodiments thereof with reference to the drawings in which like reference numerals designate identical or corresponding elements in each of the several views. These example embodiments are described so that this disclosure will be thorough and complete, and will fully convey the scope of the disclosure to those skilled in the art. Features from one embodiment or aspect can be combined with features from any other embodiment or aspect in any appropriate combination. For example, any individual or collective features of method aspects or embodiments can be applied to apparatus, product, or component aspects or embodiments and vice versa. The disclosure may be embodied in many different forms and should not be construed as limited to the embodiments set forth herein; rather, these embodiments are provided so that this disclosure will satisfy applicable legal requirements. As used in the specification and the appended claims, the singular forms “a,” “an,” “the,” and the like include plural referents unless the context clearly dictates otherwise. In addition, while reference may be made herein to quantitative measures, values, geometric relationships or the like, unless otherwise stated, any one or more if not all of these may be absolute or approximate to account for acceptable variations that may occur, such as those due to manufacturing or engineering tolerances or the like.

As used herein, “antegrade flow” refers to physiologically normal flow of fluid through the human body. In addition, “retrograde flow” refers to flow physiologically abnormal flow through the human body. Further, “patient” refers to a recipient of perfusion or retroperfusion. As used herein, “practitioner”, “user”, or “clinician” refers to an operator of a pump. In addition, the term “proximal” refers to the portion of the device or component thereof that is closer to the clinician and the term “distal” refers to the portion of the device or component thereof that is farther from the clinician.

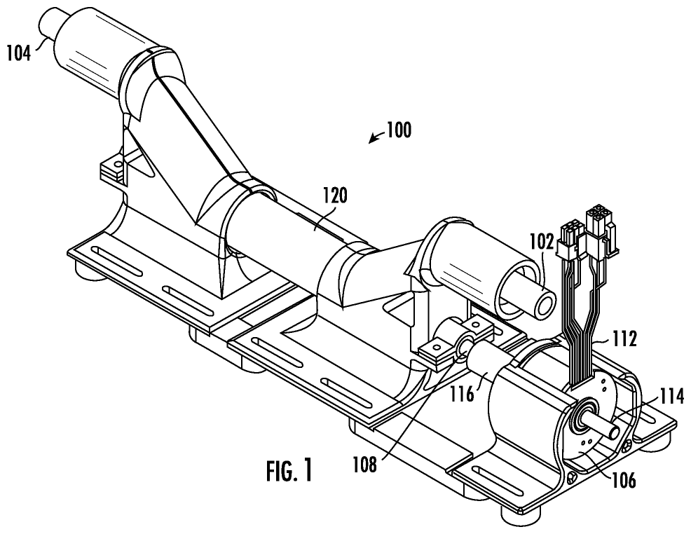

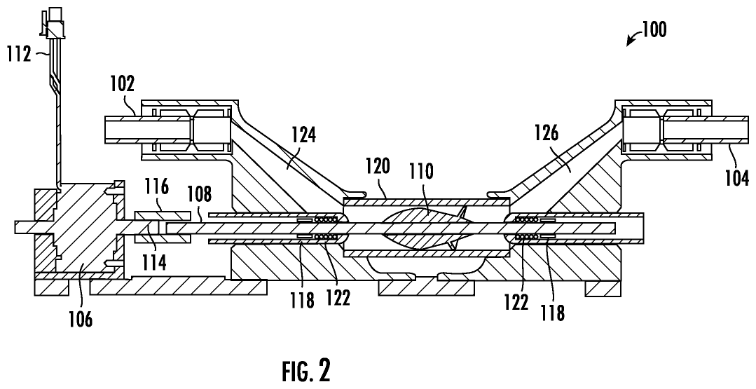

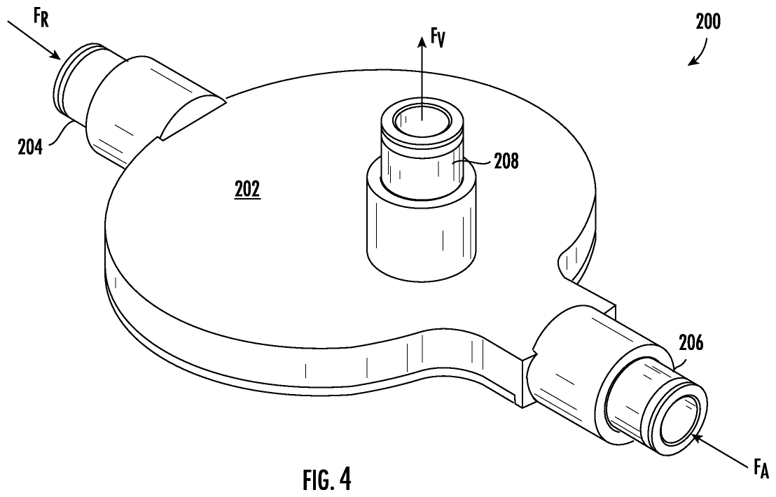

FIG. 1 is an isometric view of a bi-directional blood pump 100, according to aspects of the disclosure. FIG. 2 is a sectioned side view of pump 100, according to aspects of the disclosure. Referring to FIGS. 1 and 2, a pump 100 includes a connector 102, a connector 104, a motor 106, and an impeller shaft 108. When the pump 100 is operated for perfusion or antegrade flow, the connector 102 is an inlet that receives blood from a vein of a patient. Blood is drawn into the pump 100 by rotation of an impeller shaft 108, which includes an impeller 110 (see FIGS. 2 and 4A-4E). Blood exits the pump 100 via the connector 104 at a pressure approximately equal to the pressure of an artery of the patient to which the pump 100 is connected. For example, the pressure of blood exiting the pump 100 operating for perfusion may be between 100 mmHg-140 mmHg. The volumetric flowrate of the pump 100 operating for perfusion may be between 2.8 L/min.-3.5 L/min. In an example aspect, a tube connects to a vein of a patient to the connector 102 and a tube connects to an artery of a patient to the connector 104.

The flow characteristics of the pump 100 may be expressed in terms of hydraulic power, hydraulic power being the product of volumetric flowrate and pressure. For example, the pump 100 operates at a first pump speed with the impeller 110 rotating in a first direction and generates flow at a first hydraulic power. When the pump 100 operates at the first pump speed with the impeller 110 rotating in a second direction, opposite the first direction, the pump 100 generates flow at a second hydraulic power. In some embodiments, the first hydraulic power may be greater than the second hydraulic power. In embodiments, the first hydraulic power may correspond to antegrade flow, and the second hydraulic power may correspond to retrograde flow.

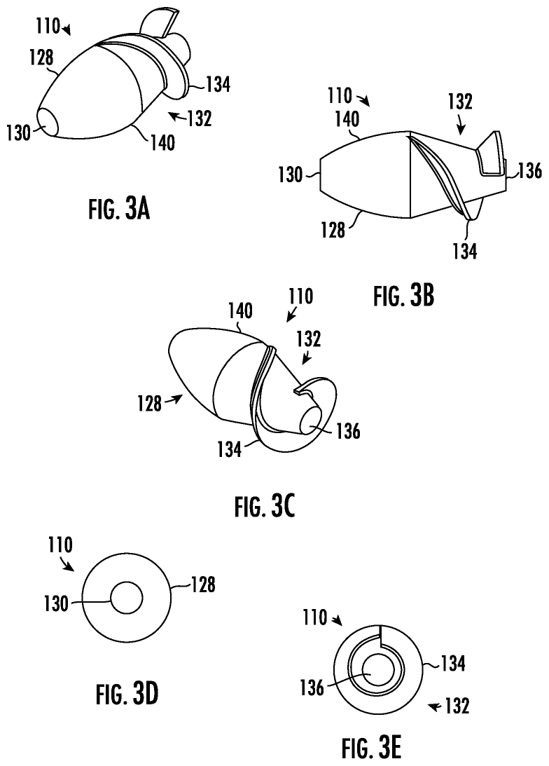

When the pump 100 is operated for retroperfusion, blood flows through the pump 100 in the reverse direction or retrograde flow and the connector 104 is an inlet that receives blood from the artery of the patient and the connector 102 is an outlet that directs blood to the vein of the patient. When operated for retroperfusion, the pressure output to the vein is approximately equal to the pressure of the vein with the impeller shaft 108 operating at the same speed as during perfusion, albeit in the opposite direction. In other words, when rotating a in first direction, the impeller 110 outputs blood having a flow rate and a pressure that is approximately equal to the natural flow rate and pressure of the artery, and, when rotating in an opposite second direction, the impeller 110 outputs blood having a flow rate and a pressure that is approximately equal to the natural flow rate and pressure of the vein. Specifically, operating the pump 100 for retroperfusion at the same pump speed results in retrograde flow having a pressure and a flow rate 30%-50% of the antegrade flow rate values of the pump 100 operating for perfusion. For example, the pressure of blood exiting the pump 100 operating for retroperfusion may be 2 mmHg-10 mmHg and the flow rate may be 1.2 L/min.-1.5 L/min. This unique behavior is enabled by the design of the impeller 110, which is discussed in more detail relative to FIGS. 3A-3E below.

Retroperfusion is not physiologically normal but provides some perfusion and the ability to control organ and/or tissue temperature. Tissue and organ temperature regulation is an additional characteristic of perfusion discussed above. In some applications, retrograde perfusion may be used to flush emboli and other debris from within vasculature. Retrograde perfusion may provide better blood distribution in the setting of chronic or acute blockages in the arterial circulation. The most common acute blockages being thromboemboli. Retroperfusion may be performed, to remove emboli from the patient with improved results as compared to perfusion.

Either antegrade or retrograde perfusion may be utilized to selectively perfuse an organ or tissue with drugs, e.g., anticoagulants, thrombolytic, agents to improve cellular function, or chemotherapeutic agents for regional cancer therapy. This includes isolated malignancies within the organ(s) being perfused.

The motor 106 is coupled to impeller shaft 108 and is configured to drive the impeller shaft 108 bi-directionally (e.g., clockwise for perfusion and counterclockwise for retroperfusion, though the opposite configuration is also contemplated depending on orientation of the vane of the impeller). The motor 106 is coupled to a controller via leads 112, which provide power to the motor 106 to control its operating parameters (e.g., speed, torque, etc.). The motor 106 includes an output shaft 114 that is coupled to an impeller shaft 108 via a coupling 116. In some aspects, the output shaft 114 and/or the impeller shaft 108 may include a keyway or the like to rotationally couple these components together. The impeller shaft 108 is supported by bearings 118 and includes the impeller 110. The impeller shaft 108 extends through an impeller housing 120 in which the impeller 110 sits. Seals 122 seal the impeller housing 120 to prevent blood from leaking from where the impeller shaft 108 enters the impeller housing 120.

A connector 102 is in fluid communication with impeller housing 120 via a conduit 124 and a connector 104 is in fluid communication with the impeller housing 120 via a conduit 126. During perfusion, blood flows from the connector 102 through the conduit 124 and into the impeller housing 120. The impeller 110 forces blood from the impeller housing 120 at a pressure that is approximately equal to that of the artery that the pump 100 is connected to. Blood then flows through the conduit 126 and exits through the connector 104.

FIGS. 3A-3E illustrate multiple views of the impeller 110, according to aspects of the disclosure. The impeller 110 includes a body 140 having a first segment 128 and a second segment 132. The first segment 128 is relatively egg-shaped with a planar face 130. The second segment 132 is frustoconical in shape with a vane 134 that spirals therearound. In some embodiments, the impeller 110 may include a plurality of vanes 134. The vane 134 is designed to provide a first flow rate and pressure when operated in a first direction (e.g., clockwise) and a second flow rate and pressure when operated in a second direction (e.g., counterclockwise). In aspects, the first pressure is 2 mmHg-10 mmHg and the second pressure is 100-140 mmHg. In some aspects, the first flowrate is 1.2 L/min.-1.5 L/min. and the second flowrate is 2.8 L/min.-3.5 L/min. The first pressure and the second pressure, and the first flowrate and the second flowrate may be achieved by the pump 100 operating at a single pump speed, but with the impeller 110 rotating in opposite directions. The pump speed may be in the range of 4,000 RPM-8,000 RPM, e.g., 6,000 RPM. The different flow characteristics are made possible by the design of impeller 110.

As best seen in FIGS. 3A-3C, the vane 134 may have a rake such that the vane 134 is angled in the axial direction so that radially outer parts of vane 134 are closer to a planar face 136 than radially inner parts of vane 134. The rake angle may be in a range of 30 degrees to 65 degrees, e.g., 48.6 degrees, relative to the central axis of the impeller 110. The rake of the vane 134 relative to central axis of the impeller 110 may be constant or variable. The rake of the vane 134 may be greater at axially lateral portions of the vane 134 and lesser at axially medial portions of the vane 134. The vane 134 may protrude from the second segment 132 at variable radial lengths. At axially medial points, the vane 134 may protrude a lesser radial length from the second segment 132 and at axially lateral points, the vane 134 may protrude a greater radial length from the second segment 132. For example, a leading edge of the vane 134, positioned medially along the body of the impeller 110, may have a lesser or greater rake relative to the central axis of the impeller 110 and protrude radially a lesser or greater length from the segment of the impeller 110 than a trailing edge of the vane 134, positioned laterally along the body of the impeller 110. In some embodiments, the rake and the length of the radial protrusion of the leading edge and the trailing edge of the vane 134 may be switched. The vane 134 may wrap helically about the body of the impeller 110 with a constant pitch or with a variable pitch. For example, the vane 134 at a leading edge may have a pitch in the range of 1-6 millimeters, e.g., 4 millimeters, and at a trailing edge may have a pitch in the range of 50-80 millimeters, e.g., 64 millimeters.

It is contemplated that other means to achieve bi-direction flow may be possible. For example, by way of control systems designed to rotate the impeller at different speeds for antegrade and retrograde flow. This may be achieved by using a standard blood pump and modulating the shaft speed based on the flow direction either continuously, or by switching among discrete speed set points. Speed modulation could either be passive, e.g., encoded in mechanisms, circuitry, or programming. Alternatively, speed modulation may be active. Active speed modulation can either be open loop, such as controlled via switches or dials, or closed loop, for example deriving feedback from flow and pressure sensors.

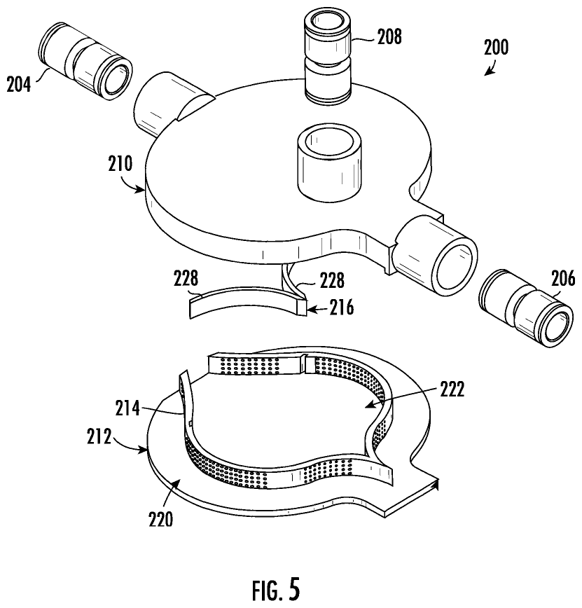

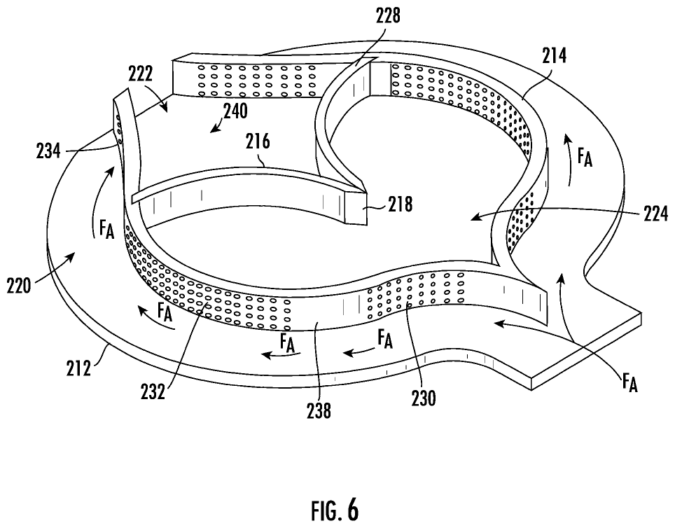

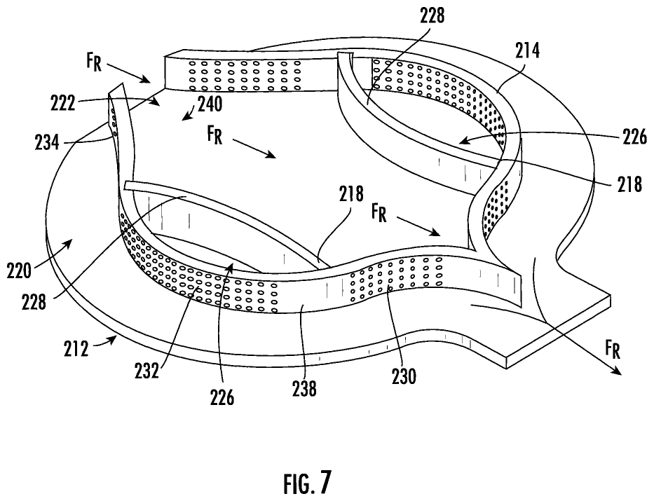

FIG. 4 is an isometric view of a filter trap 200, according to aspects of the disclosure. FIG. 5 is an exploded assembly of filter trap 200, according to aspects of the disclosure. FIG. 6 is an isometric view of a filter trap 200, according to aspects of the disclosure. FIG. 7 is an isometric view of the filter trap 200, according to aspects of the disclosure. In some aspects, the filter trap 200 may be used in combination with the pump 100. In other aspects, the filter trap 200 may be used with other equipment. Arrow FR shows the direction of retrograde flow through the filter trap 200. Arrow FA shows the direction of antegrade flow through the filter trap 200. Arrow Fv shows the direction of vacuum flow out of the filter trap 200.

The filter trap 200 includes a housing 202 and connectors 204, 206, and 208. Connectors 204 and 206 attach the filter trap 200 to tubes carrying, for example, blood. In some aspects, the filter trap 200 is placed inline with the pump 100, with the connector 206 coupled via a tube to the connector 204 and the connector 206 connected to an artery of the patient. The connector 208 is an outlet that allows, for example, emboli that have entered the housing 202 to be removed from blood flowing through the filter trap 200.

In the aspects of FIGS. 4-6, the housing 202 is formed by a first half body 210 and a second half body 212. The body 210 includes the connectors 204, 206, and 208. The body 212 includes a perforated wall 214 and a one-way valve 216. The perforated wall 214 includes a plurality of perforations 230, 232, 234 that are sized to let blood flow through but to prevent the passage of emboli. For example, the perforations 230, 232, 234 may have a diameter in a range of 50 μm to 100 μm. The perforated wall 214 may have first perforated portions 230, second perforated portions 232, third perforated portions 234, and unperforated portions 238. In some embodiments, the entire surface of the perforated wall 214 includes perforations. The perforated wall 214 partitions the housing 202 into an exterior chamber 220 formed between the walls of the housing 202 and an outer side of the perforated wall 214, and an interior chamber 222.

The valve 216 is formed from two flexible members 228 that each may include a wall engagement portion 218. The flexible members 228 of the valve 216 may each be self-biased toward the closed position of the valve 216 as shown in FIG. 6. The flexible members 228 are urged apart when flow is from connector 204 to 206 and that are urged toward the closed position when flow is from connector 206 to 204 as shown in FIG. 7. The two flexible members 228 of the valve 216 attach to the perforated wall 214 within the interior chamber 222 and further define a trap house 224 and an inlet section 240 therein. The valve 216 may be made from silicone rubber, polycarbonate, or the like. This configuration may be helpful for trapping emboli that have been dislodged from arteries during retroperfusion. For example, an emboli may be dislodged from an artery during retroperfusion and flow with the blood into the filter trap 200, entering via the connector 204. With blood flowing from the connector 204 to the connector 206 during retroperfusion, emboli enter the filter trap 200, flow past the valve 216 (which is open due to the flow), and eventually settle on the perforated wall 214 as the emboli are larger than the perforations in the perforated wall 214. Once the pump 100 is turned off and flow stops, the valve 216 will close via its self-biasing and any emboli in the filter trap 200 are trapped inside. These emboli can be suctioned out via the connector 208. However, even if the emboli are not removed and perfusion is performed, the emboli will not be able to leave the filter trap 200 as the valve 216 is in the closed position during perfusion. Blood is still able to pass through the filter trap 200 and simply passes through the perforated wall 214 and out through the connector 204.

The filter trap 200 is configured to collect a large volume or quantity of emboli such that when fluid flow through the first perforated portions 230 and the second perforated portions 232 of the perforated wall 214 is impeded, fluid may continue to flow through other portions of the third perforated portions 234. Such flow bypasses the trap house 224 in favor of the exterior chamber 220 to maintain flow parameters. During retrograde flow, the flexible members 228 of the valve 216 may be urged by the flow such that the wall engagement portion 218 engages the first unperforated portion 238 of the perforated wall 214 such that flow through the second perforated portions 232 is limited. With the wall engagement portion 218 of the flexible members 228 engaged with the unperforated portion 238, the flexible members 228 may form trap rooms 226. The trap rooms 226 may trap emboli within the trap house 224 during retrograde flow F_{R}.

In some aspects, the pump 100 may switch between antegrade flow F_{A} and retrograde flow FR multiple times during a single operation. In some instances, the filter trap 200 may have collected emboli in the trap house 224 during previous periods of retrograde flow F_{R}. Where emboli remain in the trap house 224, antegrade flow F_{A} may back flush the first perforated portions 230 such that emboli trapped against the first perforated portions 230 are dislodged from the first perforated portions 230 and move towards the flexible members 228 of the valve 216 in the closed position and/or the second perforated portions 232. Returning to retrograde flow F_{R} from antegrade flow F_{A} may cause the flexible members 228 of the valve 216 to capture previously trapped emboli between the flexible members 228 of the valve 216 and the perforated wall 214 in a trap room 226 defined between the flexible members 228 and the section of the perforated wall 214 defining the third perforated portions 234. In some embodiments, the switch from retrograde flow F_{R} to antegrade flow F_{A} may be interrupted by a pause in flow to allow the flexible members 228 of the valve 216 to self-bias themselves back to the closed position. Providing a pause in flow between retrograde flow F_{R} and antegrade flow F_{A} may prevent release of captured emboli.

In some instances, emboli may be dislodged by perfusion. In such instances, emboli may be captured in the third perforated portions 234 of the perforated wall 214. When retrograde flow FR is started, the angle of the third perforated portions 234 of the perforated wall 214 may cause the flow to pass the third perforated portions 234 in favor of a direct flow through the valve 216 to urge the wall engagement surfaces 218 of the valve 216 to contact the unperforated portion 238 of the perforated wall 214 such that flow is directed through the first perforated portions 230 of the perforated wall 214. As such, during retrograde flow F_{R} flow may be limited through the third perforated portions 234 and prevented through second perforated portions 232. The trap rooms 226 defined between the flexible members 228 of the valve 216 and the perforated wall 214 may trap emboli from previous bouts of antegrade flow F_{A}.

The filter trap 200 may include seals or gaskets to better create a fluid tight seal. The housing 202 may have two-part construction as shown or may have a unitary construction. The bodies 210 and 212 of the housing may be fused together by any suitable method, for example ultrasonic welding or bonding by adhesives.

The filter trap 200 may be used with or without the bi-directional pump 100, for purposes including, but not limited to, collecting thromboemboli, other embolic material, non-embolic in situ thrombus, or other debris. Relevant diseases when the filter trap 200, with or without the pump 100, may be used, include but are not limited to: acute myocardial infarction/coronary syndrome, acute aortic occlusion, atheroembolization, thrombotic/embolic stroke, pulmonary embolism, or deep venous thrombosis.

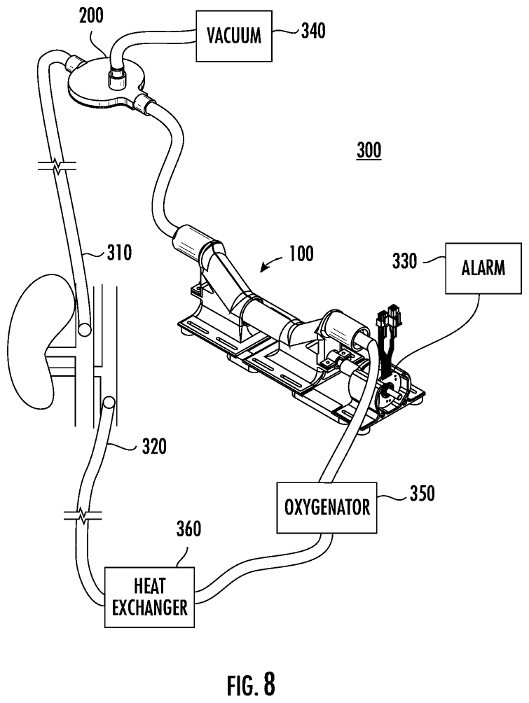

FIG. 8 is a view of a blood pump system 300, according to aspects of the present disclosure. The blood pump system 300 includes the bi-directional blood pump 100, an arterial cannula 310, and a vascular cannula 320. The blood pump system 300 may include the filter trap 200, an alarm 330, a vacuum 340, a gas exchanger or oxygenator 350, and a heat exchanger 360.

The arterial cannula 310 fluidly couples to the bi-directional blood pump 100 at the connector 104 and the vascular cannula 320 fluidly couples to the bi-direction blood pump 100 at connector 102 as described above. The filter trap 200 may spliced inline with the bi-directional blood pump 100 between the arterial cannula 310 and connector 104. The filter trap 200 operates in the blood pump system 300 as described above, with the one-way valve 216 opening only during retroperfusion.

An alarm 330 may connect to the bi-directional blood pump 100 and configured to sound or alert in response to the impeller 110 operating outside a set range of rotational speeds or outside a limit of a desired rotational speed, e.g., between 4,000 and 8,000 RPM, or 5,000 RPM±500 RPM, or 5,000 RPM±10%. When setting the alarm 330, only one operational speed value needs to be set for both perfusion and retroperfusion. Setting the alarm 330 at a singular alarm point for both perfusion and retroperfusion is due to the bi-directional blood pump 100 operating at the same rotational speeds for both perfusion and retroperfusion. The alarm 330 may be configured to alert in response to a change in pressure or a change in flowrate beyond desirable parameters. In embodiments, the alarm 330 may be coupled to a pressure sensor to measure flow pressure through a blood vessel. For example, the alarm 330 may sound when the pressure falls below 100 mmHg or exceeds 140 mmHg during antegrade flow. In embodiments, the alarm 330 may be coupled to a flow meter to measure flowrate through a blood vessel. For example, the alarm may sound when flowrate falls below 2.8 L/min. or exceeds 3.5 L/min. in antegrade flow. The alarm 330 may be configured to alert a practitioner with an audible sound, visual indica, haptic feedback, or any other appropriate indicator.

The blood pump system 300 may include a vacuum 340. The vacuum 340 may fluidly couple to the filter trap 200 at connector 208. The vacuum 340 may allow for removal of emboli captured within the filter trap 200 during retroperfusion by creating a vacuum flow Fᵥ. Removal of the debris from the filter trap 200 during operation of the pump 100 may allow for extended perfusion operations, or removal of a large amount of debris from a patient. Removal of collected debris from the filter trap 200 may allow for better flow through the pump 100 and aid in maintenance of the flowrate and pressure output by the pump 100.

A gas exchanger or oxygenator 350 may be included in the blood pump system 300 to replenish perfused or retroperfused blood with oxygen. The oxygenator 350 may be added inline with bi-directional blood pump 100. Inclusion of an oxygenator 350 may be beneficial when performing perfusion for an extended duration.

The blood pump system 300 may include a heat exchanger 360 to regulate perfusate temperature. Regulating temperature of the perfusate may prevent thermal damage to tissue and organs and may prevent the patient from become hypothermic or hyperthermic. The heat exchanger 360 may heat or cool the perfusate. For example, for short duration perfusion the bi-directional blood pump 100 may act as a heat sink resulting in a drop in blood temperature below a desirable temperature for perfusion. Conversely, for long duration perfusion the bi-directional blood pump 100 may become warm due to extended operation and heat the perfused blood above a desired temperature.

The above-mentioned accessories of the blood pump system 300, such as an oxygenator 350, may alter the flow rate and pressure characteristics of pump 100. Such accessories may necessitate changes in the operational speed of the pump 100. For example, inclusion of an oxygenator 350 may necessitate an increase in the operational speed of the pump 100, e.g., by 50 RPM.

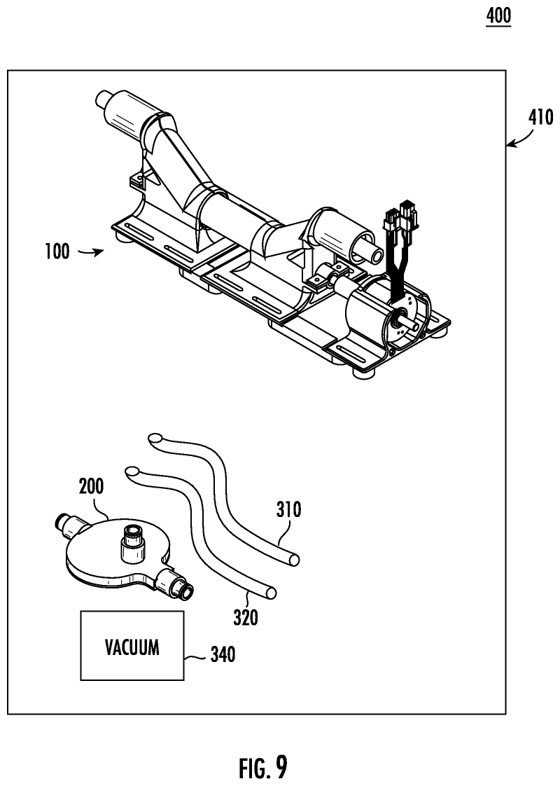

FIG. 9 is a view of a blood pump kit 400 in accordance with the present disclosure. The kit 400 may be sterilized and provide to a clinician such that it may be opened within a surgical theater or sterile field near a patient for use. The kit 400 includes the pump 100, the filter trap 200, the arterial cannula 310, the vascular cannula 320, and the vacuum 340. The kit 400 may include the alarm 330, the oxygenator 350, and/or the heat exchanger 360. The components of the kit 400 may be hermetically sealed by any appropriate means, for example in plastic wrapping 410. The components of the kit 400 may be hermetically sealed together or individually. The kit 400 may be assembled and operated as described above in relation to the blood pump system 300.

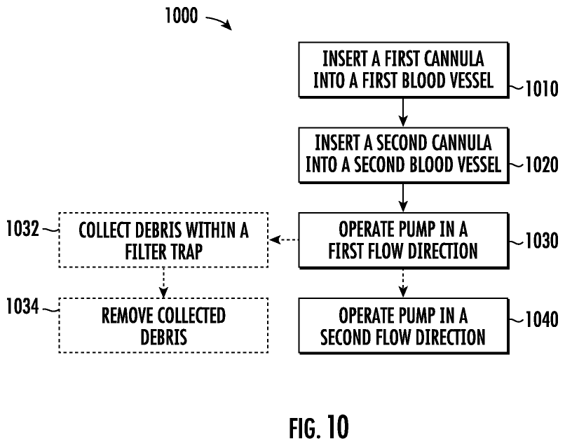

FIG. 10 is a flowchart showing a method of pumping blood in accordance with aspects of the present disclosure. The method 1000 includes inserting a first cannula into a first blood vessel, e.g., an artery, (Step 1010) and inserting a second cannula into second blood vessel, e.g., a vein (Step 1020). With the first cannula and the second cannula inserted into a first blood vessel and a second blood vessel, the bi-directional blood pump 100 may generate blood flow in a first direction (Step 1030). The direction of the flow may be selectively switched to a second flow direction (Step 1040). For example, the bi-directional blood pump 100 may selectively switch between operating in antegrade flow and operating in retrograde flow. Generating blood flow in the first direction (Step 1030) may be either antegrade flow or retrograde flow and generating blood flow in the second direction (Step 1040) may be either antegrade flow or retrograde flow. The pump 100 may operate in only one direction for an entire procedure or may operate in both the first (Step 1040) and the second direction (Step 1040) In some embodiments, the filter trap 200 may be spliced inline with bi-directional rotary pump 100. When operating in retrograde flow, the filter trap 200 may collect debris (Step 1032), for example thromboemboli, dislodged during retroperfusion. When the filter trap 200 is included, the method may include removal of collected debris from the filter trap 200 (Step 1034). In certain embodiments, a vacuum 340 may be coupled to the filter trap 200 to remove debris collected therein (Step 1034). The vacuum 340 may operate continuously or intermittently during retrograde flow (Step 1034).

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed is:

1. A blood pump system switchable between a first flow direction and a second flow direction, the blood pump system comprising:
a filter trap; and
a blood pump comprising:
a pump housing comprising:
a first connector configured as a fluid inlet into the pump housing in the first flow direction and as a fluid outlet from the pump housing in the second flow direction; and
a second connector in fluid communication with the filter trap, the second connector configured as a fluid inlet into the pump housing in the second flow direction and as a fluid outlet from the pump housing in the first flow direction; and
an impeller disposed within the pump housing, the impeller configured to rotate at a predetermined pump speed in a first rotary direction to generate fluid flow in the first flow direction at a first pressure and a first flowrate, the impeller configured to rotate in a second rotatory direction, opposite the first rotary direction, at the predetermined pump speed to generate fluid flow in the second flow direction at a second pressure and a second flowrate, the first flowrate greater than the second flowrate and the first pressure greater than the and the second pressure.

2. The blood pump system according to claim 1, wherein the impeller comprises:
a body having a first segment and a second segment; and
a vane extending radially outward from the body and helically wrapped about the second segment of the body.

3. The blood pump system according to claim 2, wherein the vane has a rake such that radially outer parts of the vane are farther from the first segment than radially inner parts of the vane.

4. The blood pump system according to claim 2, wherein the vane helically wraps about the second segment of the body with a variable pitch such that parts of the vane closer to the first segment have a lesser pitch than parts of the vane farther from the first segment.

5. The blood pump system according to claim 2, wherein the first segment is substantially egg-shaped and the second segment is frustoconically shaped.

6. The blood pump system according to claim 1, wherein the filter trap includes a one-way valve configured to remain closed when fluid flows through the pump housing in the first flow direction and to open when fluid flows through the pump housing in the second flow direction.

7. (canceled)

8. The blood pump system according to claim 1, wherein a product of the first flowrate and the first pressure is different from a product of the second flowrate and the second pressure.

9. (canceled)

10. The blood pump system according to claim 6, wherein the filter trap comprises:
a housing;
a first trap connector;
a second trap connector;
a perforated wall disposed within the housing, the perforated wall defining an interior chamber and an exterior chamber within the housing; and
the one-way valve segregating the interior chamber into an inlet section and a trap house.

11. (canceled)

12. The blood pump system according to claim 10, wherein the one-way valve comprises two flexible members attached to the perforated wall, the two flexible members configured to engage each other in a closed position and to separate from each other in an open position.

13. The blood pump system according to claim 12, wherein the two flexible members are self-biased towards to the closed position.

14. The blood pump system according to claim 12, wherein the perforated wall includes a first perforated portion and a second perforated portion, the first perforated portion separated from the second perforated portion by an unperforated portion.

15. The blood pump system according to claim 14, wherein the two flexible members each include a wall engagement portion configured to engage the unperforated portion when the two flexible members are in the open position.

16. The blood pump system according to claim 15, wherein when each flexible member is in the open position and engaged with the unperforated portion of the perforated wall, each flexible member defines a trap room with the perforated wall.

17. The blood pump system according to claim 16, wherein the second perforated portion is segregated from the trap house within a respective trap room by the flexible members.

18. The blood pump system according to claim 17, wherein fluid flow through the filter trap in the second flow direction is impeded from flowing through the second perforated portion by the flexible members engaged with the unperforated portion.

19. The blood pump system according to claim 14, wherein the perforated wall includes a third perforated portion configured to place the exterior chamber and the inlet section in direct fluid communication.

20. (canceled)

21. (canceled)

22. The blood pump system according to claim 1, further comprising a first tube fluidly coupled to the pump housing about the first connector and a second tube fluidly coupled to the pump housing about the second connector, the first tube configured to fluidly couple to a first blood vessel of the human body and the second tube configured to fluidly couple to a second blood vessel of the human body.

23. (canceled)

24. (canceled)

25. (canceled)

26. (canceled)

27. The blood pump system according to claim 1, wherein the second flowrate is between 30% and 50% of the first flowrate.

28. The blood pump system according to claim 1, wherein the first pressure is in the range of 100 mmHg to 140 mmHg and the first flowrate is in the range of 2.8 L/min. to 3.5 L/min, and the second pressure is in the range of 2 mmHg to 10 mmHg and the second flowrate is the range of 1.2 L/min. to 1.5 L/min.

29. (canceled)

30. (canceled)

31. A method of pumping blood in a first direction and a second direction, the method comprising:
inserting a first tube into a first blood vessel of a patient, the first tube fluidly coupled to a blood pump;
inserting a second tube into a second blood vessel of the patient, the second tube fluidly coupled to the blood pump;
operating the blood pump at a first operational pump speed such that blood flows through the blood pump in a first flow direction into the first blood vessel of the patient at a first pressure and a first flowrate; and
operating the blood pump at the first operational pump speed such that blood flows through the blood pump in a second flow direction into the second blood vessel at a second pressure and a second flowrate, the first flowrate greater than the second flowrate and the first pressure greater than the second pressure.
